(19) United States
(12) Patent Application Publication
(10) Pub. No.: US 2021/0206711 A1
(43) Pub. Date: Jul. 8, 2021

(54) Synthesis of the Grape Mealybug Pheromone trans-alpha-Necrodyl Isobutyrate

(71) Applicant: Trécé Inc., Tulsa, OK (US)

(72) Inventor: Vincent James Chebny, Tulsa, OK (US)

(21) Appl. No.: 17/110,909

(22) Filed: Dec. 3, 2020

Related U.S. Application Data
(60) Provisional application No. 62/956,891, filed on Jan. 3, 2020.

(51) Int. Cl. C07C 67/08; C07C 29/09; C07C 29/149; C07C 67/48; A01N 37/02; C08K 5/101

(52) U.S. Cl. CPC C07C 67/08; C07C 29/095; C08K 5/101; C07C 67/48; A01N 37/02; C07C 29/149

(57) ABSTRACT

Methods are provided for synthesizing the grape mealybug pheromone, trans-α-necrodyl isobutyrate, starting with essential plant oils that contain trans-α-necrodyl acetate. The synthesis method, in two steps, converts the trans-α-necrodyl acetate to trans-α-necrodol and then converts the trans-α-necrodol to the desired trans-α-necrodyl isobutyrate. The first step can be achieved by either of a hydrolysis reaction or a reduction reaction. In the hydrolysis reaction, the essential oil is mixed in an alcohol solvent with a hydroxide base, or by mixing the essential oil in an aqueous acid. A reduction reaction can instead by performed in an organic solvent in the presence of a reducing agent. The trans-α-necrodol is converted to the desired trans-α-necrodyl isobutyrate by an esterification reaction with a carboxylic acid, an acyl halide, or an acid anhydride.

### CROSS-REFERENCE TO RELATED APPLICATIONS

This application claims the benefit of U.S. provisional application No. 62/956,891 filed on Jan. 3, 2020, the disclosure of which is incorporated herein by reference.

### BACKGROUND

### Field of the Invention

The invention is in the field of insect monitoring and control, and more particularly in the production of synthetic insect pheromones.

### Related Art

The grape mealybug, Pseudococcus maritimus, is a destructive pest that attacks grapes as well as most deciduous fruit crops. The female-produced sex pheromone is known to be trans-α-necrodyl isobutyrate. Y. Zou et al., “Synthesis and Bioassay of Racemic and Chiral trans-α-Necrodyl Isobutyrate, the Sex Pheromone of the Grape Mealybug Pseudococcus maritimus,” University of California, Riverside, 2010 describe a complex synthesis of the pheromone starting with the production of methyl 4-bromoacetoacetate. Ethyl acetoacetate is added to the methyl 4-bromoacetoacetate to produce a semisolid product which is then mixed with acetic acid and diglyme, refluxed, extracted with ether, washed, dried, and concentrated to yield 2-methyl-4-oxo-cyclopent-2-enecarboxylic acid ethyl ester. Next, Me₂Zn is added to a suspension of the 2-methyl-4-oxo-cyclopent-2-enecarboxylic acid ethyl ester and the solution further processed through several additional steps including cooling to −78 C to yield 2,2,3-trimethyl-4-oxo-cyclopentanecarboxylic acid ethyl ester. This ethyl ester is, in turn, added to a solution including a slurry made from zinc dust and TiCl₄. Additional processing steps yields 2,2,3-trimethyl-4-methylene-cyclopentanecarboxylic acid ethyl ester. The process continues in this fashion through the synthesis of α-necrodol from the 2,2,3-trimethyl-4-methylene-cyclopentanecarboxylic acid ethyl ester, and finally trans-α-necrodyl isobutyrate from the α-necrodol.

Y. Zou et al. notes that in the production of the 2-methyl-4-oxo-cyclopent-2-enecarboxylic acid ethyl ester the relative positions of the methyl and ethyl esters were critically important; during optimization of the reaction parameters, it was found that if the methyl and ethyl ester components were switched, the decarboxylation reaction gave only a complicated mixture of products. The following step of their synthesis was also found to be critically dependent on the solvent used. Further peculiarities to the process were also noted. It was reported that the overall yield for the entire synthetic sequence was 7%.

What is needed, therefore, is a simpler synthesis method to yield trans-α-necrodyl isobutyrate, preferably with a higher yield.

### SUMMARY

The present invention provides a simplified synthesis method for producing trans-α-necrodolyl isobutyrate that advantageously starts with natural essential oils derived from plants and produces much greater yields. In various embodiments the essential oil comprises at least 0.1% trans-α-necrodyl acetate, and can comprise an essential oil of Lavender Seville, Evolvulus alsinoides L., or Lavandula luisieri. In some embodiments the essential oil further includes trans-α-necrodol.

An exemplary method of the present invention begins by preparing a first product including trans-α-necrodol from the essential oil including at least some trans-α-necrodyl acetate by either a hydrolysis reaction or a reduction reaction. Subsequently, a second product including trans-α-necrodolyl isobutyrate is prepared from the trans-α-necrodol by an esterification reaction with a carboxylic acid, an acyl halide, or an acid anhydride. The second product is optionally refined to produce a third product with a higher concentration of trans-α-necrodyl isobutyrate than in the second product. The trans-α-necrodyl isobutyrate so produced can be further formulated into a sprayable, solid, or liquid form.

As noted, in various embodiments preparing the first product including trans-α-necrodol from the essential oil includes a hydrolysis reaction. In these embodiments preparing the first product including trans-α-necrodol can include mixing the essential oil in an alcohol solvent with a hydroxide base, such that the hydrolysis reaction occurs between the hydroxide base and the trans-α-necrodyl acetate. In some of these embodiments the alcohol solvent comprises one or more of methanol, ethanol, isopropanol, propanol, isomers of butanol, and isomers of pentanol and the hydroxide base can comprise sodium hydroxide, potassium hydroxide, or lithium hydroxide.

As also noted, in various embodiments preparing the first product including trans-α-necrodol from the essential oil includes a reduction reaction. In some of these embodiments preparing the first product including trans-α-necrodol is performed in an organic solvent in the presence of a reducing agent. Here, the organic solvent can comprise ether, toluene, or hexane, for example, while the reducing agent can comprise lithium aluminum hydride, sodium borohydride, lithium borohydride, hydrogen gas, lithium hydride, sodium hydride, vitride, or diisobutylaluminum hydride. In still other embodiments in which this step includes a hydrolysis reaction, preparing the first product including trans-α-necrodol includes mixing the essential oil in an aqueous acid, such as sulfuric acid, hydrochloric acid, or acetic acid.

In various embodiments, in the step involving the esterification reaction, the acyl halide can optionally comprise isobutryl chloride, the acid anhydride can optionally comprise isobutyric anhydride, the carboxylic acid can optionally comprise isobutyric acid, and the amine base can optionally comprise pyridine or triethylamine.

### BRIEF DESCRIPTION OF THE DRAWINGS

FIG. 1 is a flowchart representation of a method for synthesizing the grape mealybug pheromone according to various embodiments of the present invention.

FIG. 2 illustrates a first reaction step according to various embodiments of the present invention.

FIG. 3 illustrates a second reaction step according to various embodiments of the present invention.

### DETAILED DESCRIPTION

The present invention is directed to a method of synthesizing trans-α-necrodyl isobutyrate (trans-(3,4,5,5-Tetramethylcyclopent-2-en-1-yl)-methyl 2-methylpropanoate)) starting with essential plant oils that contain trans-α-necrodyl acetate and that may also already contain some amount of trans-α-necrodol ((3,4,5,5-Tetramethylcyclopent-2-enyl)methanol). The synthesis method, in two steps, converts trans-α-necrodyl acetate to trans-α-necrodol and then converts the trans-α-necrodol to the desired trans-α-necrodyl isobutyrate. As compared to the prior synthesis of Y. Zou et al., the method disclosed herein involves far fewer steps, begins with inexpensive and readily available materials, provides much higher yields, and is scalable to larger produced quantities.

An essential oil of a plant, as used herein, is a concentrated hydrophobic liquid containing volatile (easily evaporated at normal temperatures) chemical compounds derived from that particular plant. An essential oil is ‘essential’ in the sense that it contains the ‘essence of’ the plant's fragrance—the characteristic fragrance of the plant from which it is derived; ‘essential’ as used herein expressly does not mean indispensable.

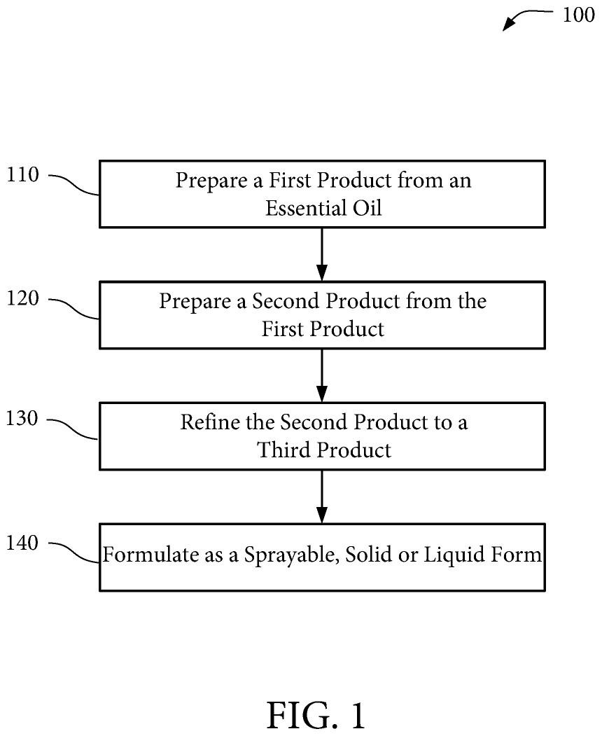

FIG. 1 is a flowchart representation of an exemplary method 100 for synthesizing the grape mealybug pheromone. The method 100 comprises a first step 110 of preparing a first product from an essential oil including trans-α-necrodol acetate. In a step 120, a second product is prepared from the first product by converting the trans-α-necrodol to trans-α-necrodolyl isobutyrate. In an optional step 130 the second product is refined to a third product characterized by a higher concentration of trans-α-necrodyl isobutyrate than in the second product. In an optional step 140 the third product is formulated into a form suitable for insect monitoring and control in agriculture.

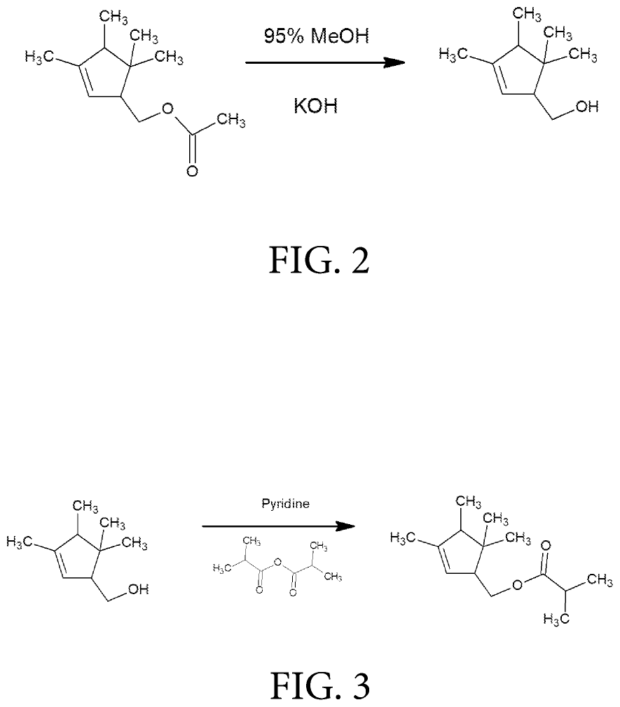

Step 110 is further illustrated by the chemical reaction shown in FIG. 2. In the step 110, an essential oil including trans-α-necrodol acetate is processed to produce a first product including trans-α-necrodol. Suitable essential oils include, for example, that from Lavender Seville, (Lavandula stoechas subsp. Luisieri (Rozeira)Rozeira), that from the slender dwarf morning-glory (Evolvulus alsinoides (L.) L.), and that from the L. stoechas luisieri subspecies of the lavender Lavandula stoechas. In various embodiments a minimum quantity of the trans-α-necrodyl acetate in the essential oil is 0.1%.

The reaction of FIG. 2, as one exemplary embodiment of the first step 110, is a hydrolysis reaction between the trans-α-necrodyl acetate in the essential oil and a hydroxide base carried out in an alcohol solvent. In the illustrated embodiment, the hydroxide base is potassium hydroxide and the alcohol solvent is methanol. Suitable hydroxide bases include, for example, sodium hydroxide, potassium hydroxide, lithium hydroxide, and mixtures thereof. Suitable alcohol solvents include methanol, ethanol, isopropanol, propanol, isomers of butanol, and isomers of pentanol, and mixtures thereof, for example.

For those embodiments in which step 110 employs a hydroxide base in an alcohol solvent, the first reaction can be carried out under an inert atmosphere such as an argon atmosphere. In various embodiments, the solvent can optionally include some water, for example, a mixture comprising 95% methanol and 5% water can be employed (percent by weight). In an exemplary embodiment, 80 g of Lavender Seville essential oil reacted with 41.9 g of KOH will yield the first product, after solvent removal, of about 60 g of a crude material, that is approximately 34% trans-α-necrodol.

In other embodiments the first step 110 comprises a reduction reaction to convert the trans-α-necrodol acetate in the essential oil to trans-α-necrodol. For instance, to carry out the reaction under reducing conditions, step 110 can be performed in an organic solvent such as ether, toluene, hexane, or mixtures thereof, in the presence of a reducing agent like lithium aluminum hydride, sodium borohydride, lithium borohydride, hydrogen gas, lithium hydride, sodium hydride, vitride, diisobutylaluminum hydride, or combinations thereof. In various embodiments the reducing agent is first added to the organic solvent, followed by the essential oil.

In some embodiments in which the first reaction is a hydrolysis reaction, the reaction is carried out under acidic conditions to convert the trans-α-necrodol acetate in the essential oil to trans-α-necrodol. For instance, to carry out the reaction under acid hydrolysis conditions, step 110 can be performed in the presence of an acid like hydrochloric acid, sulfuric acid and acetic acid. In these embodiments, preparing the second product can include mixing the essential oil in an aqueous acid, where the hydrolysis reaction occurs between the aqueous acid and the trans-α-necrodyl acetate. Suitable aqueous acids include sulfuric acid, hydrochloric acid, and acetic acid.

The first reaction in step 110 can be aided by stirring or agitation until complete. The reaction can be carried out at room temperature, in some embodiments. Higher temperatures will increase the rate of reaction.

Following the completion of the reaction, step 110 can further include separating the first product, including the trans-α-necrodol, from the solvent solution. For example, in one embodiment the combination of the solvent solution and the first product, including the trans-α-necrodol, is quenched in brine, followed by an aqueous extraction using dichloromethane. Then, organic layers are combined and washed with water, brine, and dried over magnesium sulfate. Lastly, any remaining solvent can be removed using a rotary evaporator, leaving just the first product.

Step 120 comprises preparing a second product from the first product by converting the trans-α-necrodol in the first product to trans-α-necrodolyl isobutyrate. Step 120 comprises a second reaction, that in an exemplary embodiment comprises an esterification reaction of the trans-α-necrodol with one or more of a carboxylic acid, an acyl halide, or an acid anhydride to form trans-α-necrodyl isobutyrate. The second reaction is carried out in an organic solvent such as ether, toluene, hexane, or mixtures thereof. FIG. 3 illustrates an exemplary second reaction in which isobutyric anhydride is used as the acid anhydride and the amine base is pyridine. A suitable acyl halide that can be used in other embodiments is isobutryl chloride. A suitable carboxylic acid is isobutyric acid for Fisher esterification, Steglich esterification, or Yamaguchi esterification. A suitable acid anhydride is isobutyric anhydride. Another suitable amine base is triethylamine.

Step 120 is also conducted in a suitable vessel, again such as a Schlenk flask for the synthesis of laboratory bench quantities. Preferably, the second reaction is also carried out under an inert atmosphere such as an argon atmosphere. The second reaction can also be aided by stirring or agitation until complete. The second reaction can be conducted from −78° C. to 100° C., and in some embodiments it is performed at 0° C. In an exemplary embodiment of step 120, 60 g of the first product including trans-α-necrodol from step 110 is mixed with 154.2 g of isobutyric anhydride and 94.2 ml of dry pyridine in 600 ml of ether. This second reaction yields about 62 g of the second product including the desired trans-α-necrodyl isobutyrate.

Step 120 can further include, after the second reaction is complete, in various embodiments, separating the organic solvent from the second product. In some embodiments, the combination of the second product and the organic solvent solution is quenched in cold water, layers are separated, and the organic layer is washed with 10% HCl, water, 5% KOH, and brine then dried over magnesium sulfate, and filtered. Any remaining solvent can be removed using a rotary evaporator.

In further embodiments, this material can be further refined, such as by distillation at 70° C. under a pressure of 1.5 mmHg. About 33 g of a third product including about 64% of the desired trans-α-necrodyl isobutyrate can be obtained from this distillation process starting with about 62 g of the second product. In still further embodiments the third product can be purified to essentially 100% trans-α-necrodyl isobutyrate by flash chromatography. In an exemplary embodiment, the flash chromatography is performed on 10% AgNO₃ silica gel using 0.5% ether/hexane as an eluent.

Since the process begins with an essential oil, other compounds that may be present in the second and third products include those listed in the following table:

| | |
|---|---|
| 1 | Alpha pinene |
| 2 | cis-alpha-necrodol |
| 3 | Cymene |
| 4 | Alpha Terpineol |
| 5 | 1,8-Cineole |
| 6 | Alpha Terpinyl acetate |
| 7 | Fenchone |
| 8 | Trans-alpha-necrodol |
| 9 | Camphor |
| 10 | Delta Cadinene |
| 11 | Linalool |
| 12 | Selina 3,7 (11) diene |
| 13 | Trans-alpha-necrodyl acetate |
| 14 | Viridiflorol |
| 15 | Lavandulyl Acetate |
| 16 | Methyl Eugenol |
| 17 | Cis-alpha-necrodol acetate |
| 18 | Alpha Cadinol |
| 19 | 5-Menthylene-2,3,4,4-Tetrame-2-cyclopentenone |
| 20 | cis-alpha-necrodol isobutyrate |
| 21 | Fenchyl alcohol |
| 22 | Fenchyl isobutyrate |
| 23 | linalool isobutyrate |
| 24 | Lavandulol |
| 25 | Lavandulyl isobutyrate |
| 26 | Alpha Terpineol isobutyrate |
| 27 | Viridiflorol isobutyrate |
| 28 | Alpha Cadinol isobutyrate |

The reactions described above can be conducted in any suitable vessel, such as a Schlenk flask for the synthesis of laboratory bench quantities. Larger quantities for commercial purposes can be prepared in jacketed kettles with overhead mixers.

The following NMR results provide spectral evidence of the synthesis.

¹H NMR (CDCl₃, 300 MHz): δ 5.14 (br d, J=3.7), 4.05 (dd, J=6.5, 10.6), 3.90 (dd, J=6.9, 10.8), 2.50 (m), 2.46 (m), 2.13 (m), 1.64 (t, J=1.7), 1.12 (d, J=7.0), 1.08 (d, J=6.0), 0.95 (s), 0.94 (s), 0.87 (d, J=7.3)

¹³C NMR (CDCl₃, 300 MHz): δ 177.3 (C), 145.4 (C), 123.2 (CH), 64.7 (CH2), 52.7 (CH), 52.6 (CH), 43.1 (C), 34.2 (CH), 24.7 (CH3), 24.1 (CH3), 19.13 (CH3), 19.09 (CH3), 15.3 (CH3), 12.5 (CH3).

The trans α-necrodyl isobutyrate can be used, for example, as an insect attractant for grape mealybug (Pseudococcus maritimus), and can be used to lure the grape mealybug into a trap. The trans α-necrodyl isobutyrate can also be used to disrupt the grape mealybug mating, in various embodiments. For these purposes the trans α-necrodyl isobutyrate can be formulated into a sprayable, solid or liquid form. The trans α-necrodyl isobutyrate can also be impregnated into a thermoplastic such as polyethylene, polyvinyl chloride, polypropylene, ethylvinyl acetate, polystyrene, or rubber.

In the foregoing specification, the invention is described with reference to specific embodiments thereof, but those skilled in the art will recognize that the invention is not limited thereto. Various features and aspects of the above-described invention may be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

What is claimed is:

1. A method comprising:
preparing a first product including trans-α-necrodol from an essential oil including trans-α-necrodyl acetate by a hydrolysis reaction or a reduction reaction; and
preparing a second product including trans-α-necrodolyl isobutyrate from trans-α-necrodol by an esterification reaction with a carboxylic acid, an acyl halide, or an acid anhydride.

2. The method of claim 1 wherein the essential oil comprises at least 0.1% trans-α-necrodyl acetate.

3. The method of claim 1 wherein the essential oil comprises an essential oil of Lavender Seville, Evolvulus alsinoides L., or Lavandula luisieri.

4. The method of claim 1 wherein the essential oil further includes trans-α-necrodol.

5. The method of claim 1 wherein preparing the first product including trans-α-necrodol includes mixing the essential oil in an alcohol solvent with a hydroxide base, wherein the hydrolysis reaction occurs between the hydroxide base and the trans-α-necrodyl acetate.

6. The method of claim 5 wherein the alcohol solvent comprises one or more of methanol, ethanol, isopropanol, propanol, isomers of butanol, and isomers of pentanol.

7. The method of claim 5 wherein the hydroxide base comprises sodium hydroxide, potassium hydroxide, or lithium hydroxide.

8. The method of claim 1 wherein preparing the first product including trans-α-necrodol is performed in an organic solvent in the presence of a reducing agent.

9. The method of claim 8 wherein the organic solvent comprises ether, toluene, or hexane.

10. The method of claim 8 wherein the reducing agent comprises lithium aluminum hydride, sodium borohydride, lithium borohydride, hydrogen gas, lithium hydride, sodium hydride, vitride, or diisobutylaluminum hydride.

11. The method of claim 1 wherein preparing the first product including trans-α-necrodol includes mixing the essential oil in an aqueous acid.

12. The method of claim 11 wherein the aqueous acid comprises sulfuric acid, acetic acid, or hydrochloric acid.

13. The method of claim 1 wherein the acyl halide comprises isobutryl chloride.

14. The method of claim 1 wherein the acid anhydride comprises isobutyric anhydride.

15. The method of claim 1 wherein the carboxylic acid comprises isobutyric acid.

16. The method of claim 1 wherein the amine base comprises pyridine or triethylamine.

17. The method of claim 1 further comprising refining the second product to produce a third product with a higher concentration of trans-α-necrodyl isobutyrate than in the second product.

18. A product made by the process of claim 1.

19. The method of claim 1 further comprising formulating the trans-α-necrodyl isobutyrate into a sprayable, solid, or liquid form.

20. The method of claim 19 wherein the solid comprises a thermoplastic.

21. The method of claim 20 wherein the thermoplastic consists of polyethylene, polyvinyl chloride, polypropylene, ethylvinyl acetate, polystyrene, or rubber.
